(19) United States
(12) Patent Application Publication
(10) Pub. No.: US 2026/0232707 A1
(43) Pub. Date: Aug. 13, 2026

(54) METABOLIC INHIBITORS AND METHODS OF MAKING AND USING THEREOF

(71) Applicant: UNIVERSITY OF MIAMI, Miami, FL (US)

(72) Inventors: Shanta DHAR, Miami, FL (US); Xiao ZHANG, Miami, FL (US); Shrita SARKAR, Miami, FL (US)

(21) Appl. No.: 19/154,279

(22) PCT Filed: Feb. 8, 2024
(86) PCT No.: PCT/US2024/014980

§ 371 Date: Aug. 6, 2025

Related U.S. Application Data
(60) Provisional application No. 63/484,058, filed on Feb. 9, 2023.

(51) Int. Cl. A61K 31/695; A61K 9/51; C07F 7/04

(52) U.S. Cl. CPC A61K 31/695; A61K 9/5146; C07F 7/04

(57) ABSTRACT

Disclosed are compounds, nanoparticles, and compositions for effective delivery of metabolic inhibitors to disease state cells. The compounds, nanoparticles, and compositions disclosed herein show trackability in biological system, extended stability, and other advantageous physicochemical properties to treat various disease states. Also disclosed are methods of treating a subject in need thereof, such as a subject with cancer.

### CROSS-REFERENCE TO RELATED APPLICATIONS

This application claims benefit of priority of U.S. Provisional Application No. 63/484,053, filed Feb. 9, 2023, which is incorporated herein by reference.

### STATEMENT REGARDING FEDERALLY SPONSORED RESEARCH OR DEVELOPMENT

This invention was made with government support under Grant No. 1P30CA240139 awarded by the National Cancer Institute. The Government has certain rights in the invention.

### BACKGROUND

Cells generally have a standard or preferred metabolic pathway that they utilize to meet their energy requirement to function. Normal cells generally transform glucose into carbon dioxide and water under aerobic conditions, by means of oxidative phosphorylation. However, altered metabolic pathways may emerge when a cell enters a disease state which utilizes a less efficient means of ATP production. One such pathway is glycolysis which is exaggerated in many disease-state cells. Despite a lesser overall yield of ATP than mitochondrial respiration, glycolysis requires less energy input and can therefore be hijacked to operate at a remarkably higher rate-affording some diseased cells a survival advantage in hypoxic conditions. Amid this unique metabolic shift, recent attention has focused on the disruption of glycolysis to inhibit diseased cell proliferation and improve therapeutic efficacy in such diseases.

One of the most studied target enzymes to achieve glycolytic inhibition is pyruvate dehydrogenase kinase-1 (PDK1). PDK1 is a major inhibitory regulator of the enzyme pyruvate dehydrogenase (PDH), which is essential for the oxidative decarboxylation of glycolytic pyruvate to form acetyl-CoA for mitochondrial respiration. By targeting and inhibiting PDK1, PDH can operate in an unregulated manner, as the negative feedback inhibition loop created by acetyl-CoA is now lost. However, many PDK1 inhibitors lack effective cellular uptake and have poor localization inside the mitochondrial matrix where the PDK1 is located. This greatly increases the required dose for tumor suppression and thereby limits its therapeutic applications. Thus, improved delivery of metabolic inhibitors would be beneficial for the treatment of diseases exhibiting altered metabolic pathways. The compounds and methods disclosed herein address these and other needs.

### SUMMARY

Disclosed herein are compounds and methods of making and using thereof. In some embodiments, these compounds can function as metabolic inhibitors. These compounds can comprise a mitochondrial targeting moiety, a moiety that inhibits a metabolic process (e.g., glycolysis), and a moiety that improves the stability of the compound in vivo (e.g., a hydrophobic moiety such as a lipid chain that reduces hydrolysis in vivo). In some embodiments, the compounds can further comprise a moiety that facilitates tracking of the compound within a biological sample.

For example, disclosed herein are compounds comprising a structure represented by Formula I, or a pharmaceutically acceptable salt thereof:

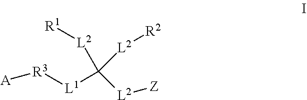

wherein
A comprises a mitochondrial targeting moiety;
Z comprises a lipid chain;
R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene, or —R^{a}X—, wherein R^{a} is a C₀-C₄ alkylene and X is a detectable-group such as silyl group or silicon or fluorophore.

In some examples, the compound comprises Formula II, or a pharmaceutically acceptable salt thereof:

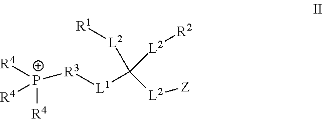

wherein
Z comprises a lipid chain;
R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
each R⁴ is independently a substituted or unsubstituted aryl or heteroaryl;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene, or —R^{a}X—, wherein R^{a} is a C₀-C₄ alkylene and X is a detectable-group such as silicon or silyl group or fluorophore.

In some examples, the compound comprises Formula III, or a pharmaceutically acceptable salt thereof:

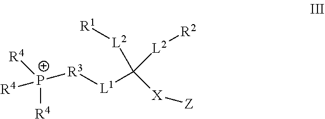

wherein
Z comprises a lipid chain;
R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
each R⁴ is independently a substituted or unsubstituted aryl or heteroaryl;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene, and
X is a detectable-group such as a silyl group.

In some examples, the compound comprises Formula IV, or a pharmaceutically acceptable salt thereof:

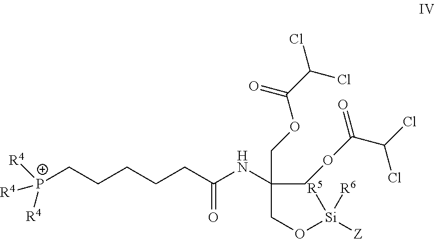

wherein
Z comprises a lipid chain;
each R⁴ is independently a substituted or unsubstituted aryl or heteroaryl; and
R⁵ and R⁶ are independently hydrogen or a C₁-C₈ alkyl.

In some examples, the compound comprises:

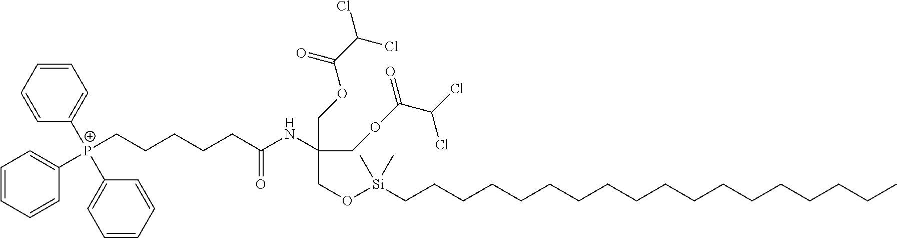 [SMILES: CCCCCCCCCCCCCCCCCC[Si](C)(C)OCC(COC(=O)C(Cl)Cl)(COC(=O)C(Cl)Cl)NC(=O)CCCCC[P+](c1ccccc1)(c1ccccc1)c1ccccc1]

or a pharmaceutically acceptable salt thereof.

Also disclosed herein is a nanoparticle (e.g., one or more nanoparticles) comprising any of the compounds disclosed herein and a polyethylene glycol-lipid.

Also disclosed herein is a pharmaceutical composition comprising a pharmaceutically acceptable carrier and an effective amount of the compounds or nanoparticles described herein.

Also disclosed herein are methods of treating a disease or disorder in a subject in need thereof, the methods comprising administering to the subject a therapeutically effective amount of any of the pharmaceutical compositions disclosed herein. In some examples, the disease comprises cancer.

### BRIEF DESCRIPTION OF THE FIGURES

The accompanying figures, which are incorporated in and constitute a part of this specification, illustrate several aspects described below.

FIGS. 1A-1D. (FIG. 1A) Chemical structures of currently available major glycolysis inhibitors. (FIG. 1B) Design of Mito-SilylDCA incorporating the -TPP moiety to access PDK1 in the mitochondrial network (designated in pink); multiple DCA moieties for attaining PDK1 inhibition (designated in green); the poly-ethylene long chain (designated in blue) to provide increased stability and hydrophobicity for incorporation in a delivery vehicle; and the silyl group (designated in red) to attain the biological detection ability. (FIG. 1C) A synthetic route to Mito-SilylDCA. The reactant, (5-carboxypentyl) triphenylphosphonium bromide was protected with tris(hydroxymethyl)aminomethane (Tris) in the presence of N-ethoxycarbonyl-2-ethoxy-1,2-dihydroquinoline (EEDQ) to selectively couple amine group with carboxylic acid in the presence of hydroxy functionality to result in Compound 1. In the next step of synthesis, chloro(dimethyl)octadecylsilane was coupled to Compound 1 in presence of triethylamine (Et₃N) to synthesize compound 2 which was further coupled with DCA-anhydride to develop the final target compound Mito-SilylDCA. The percentage values mentioned below each arrow of the synthesis process corresponds to the percent yield of the product. (FIG. 1D) Characterization of Mito-SilylDCA by 1H spectroscopy CDCl₃.

FIGS. 2A-2B. Stability of Mito-SilylDCA at different pH values. Traces of HPLC plots of (FIG. 2A) Mito-SilylDCA and (FIG. 2B) Mito-DCA in solutions of different pH values along with plots of percent of the compound remained with time. A solution of Mito-SilylDCA or Mito-DCA in DMF-water (2/1, v/v) was incubated for different times (12 h, 24 h, and 48 h) at different pH environments (4.0, 7.4, and 9.5) at room temperature. The line chart showed the stability of Mito-SilylDCA and Mito-DCA with time.

FIGS. 3A-3C show cellular glycolysis inhibition by Mito-SilylDCA. Glycolytic inhibition efficacy of Mito-SilylDCA was investigated along with the components used for synthesizing Mito-SilylDCA. (FIG. 3A) Chemical structures of TPP-(CH₂)₅—COOH), methoxyl(octadecyldimethyl)silane, sodium dichloroacetate (NaDCA), Mito-DCA, and Mito-SilylDCA. (FIG. 3B) Extracellular acidification rate (ECAR) measured in 4T1, murine breast cancer cell line, upon treatment with the compounds mentioned in A. The cells were pre-treated with the test articles at the following concentration: TPP-(CH₂)₅—COOH (200 μM), methoxy (octadecyldimethyl)silane (200 μM), NaDCA (600 μM), Mito-DCA (200 μM), or Mito-Silyl-DCA (200 μM), for 6 h. Port injections for the seahorse glycostress assay included glucose (10 mM) in port A, oligomycin (1 μM) in port B, and 2-DG (50 mM) in port C. (FIG. 3C) Graphical representation quantifying the change in glycolysis, glycolytic capacity, and glycolytic reserve parameters upon treatment with test articles. The data in FIG. 3C was generated as an average from 3 independent biological replicates. The statistical significance between the treatment groups was measured by ordinary one-way analysis of variance (ANOVA) using multiple comparison.

FIGS. 4A-4E show characterizations of T-Mito-SilylDCA-NPs. (FIG. 4A) Graphical representation of T-MitoSilyl-DCA-NPs showing structures of polymers and ligands. Changes in (FIG. 4B) hydrodynamic diameter (nm) and zeta potential (mV). (FIG. 4C) % loading and (FIG. 4D) % encapsulation efficiency of Mito-SilylDCA as measured by ICP-MS and HPLC. A varying % feed of T-Mito-SilylDCA-NPs were synthesized starting from 10% to 50%. ICP-MS was used to quantify the silicon (Si) and HPLC was used to -TPP in T-Mito-SilylDCA-NPs. (FIG. 4E) TEM images 20% Mito-SilylDCA feed particles reveal the spherical morphology of the T-Mito-SilylDCA-NPs.

FIGS. 5A-5G show in vivo efficacy of T-Mito-SilylDCA-NPs utilizing a breast cancer xenograft model using MDA-MB-231BR cells with metastatic potential. (FIG. 5A) Schematic representation showing the implantation of MDA-MB-231BR^{Luc} breast cancer cells with metastatic potential in the right flank of N=7 female BALB/c nude mice via subcutaneous injection. The illustration demonstrates the treatment regimen using 10 mg/kg of T-Platin-M-NPs with respect to Platin-M to alleviate the glycolytic pathways in the tumor followed by treatment with 30 mg/kg of T-Mito-Silyl-DCA-NPs with respect to Mito-DCA to show inhibition of this metabolism. (FIG. 5B) Kaplan-Meier survival curve showing the significantly increased survival of mice that were treated with these nano-formulations. Scatter plot showing the change in (FIG. 5C) body weight (g) and (FIG. 5D) tumor volume (mm³) across the treatment regimen. (FIG. 5E) Brightfield images of n=3 mice from saline group and n=4 mice from treated group showing the differences in tumor growth from Day 0 to Day 12 post cell inoculation. All images were taken keeping the field of view constant at 22.4 cm (FIG. 5F) Biodistribution data showing the presence of Mito-Silyl-DCA in liver, tumor mass, brain, kidney, and spleen. (FIG. 5G) Immunofluorescence study showing inhibition of glycolytic markers HKII and PDK1, and upregulation of apoptosis marker, caspase9. The scale bar for the immunofluorescence images are 40 μm.

FIGS. 6A-6C. (FIG. 6A) Nanoparticle diameter and surface charge variation with different percent feed of Mito-SilylDCA. Percent loading and encapsulation efficiency by (FIG. 6B) ICP-MS by quantifying Si and (FIG. 6C) by HPLC.

### DETAILED DESCRIPTION

The materials, compounds, compositions, articles, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

In general, the number of carbon atoms present in a given group may be designated as a range from “Cₓ-C_{y}”, where x and y are the lower and upper bounds of the range, respectively. The carbon number as used in the definitions herein refers to carbon backbone and carbon branching, but does not include carbon atoms of the substituents, such as alkoxy substitutions and the like. In some instances, a group may be referenced as including C₀, which means that the group is absent or, in the case of a linking group, represents a direct bond whereby the groups at both ends of the linking group are directly linked. The term “direct bond,” as used herein, refers to an embodiment where the identified group is absent from the structure, and is replaced by a bond between other groups to which it is connected. For example, if the specification or claims recite A-D-E and D is defined as a direct bond, the resulting structure is A-E.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O, which is also referred to herein as a “carbonyl.”

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “carbamide” means compounds having the group —N(Z¹)—(CO)N(Z¹)₂ where each Z¹ can be, independently, an alkyl, alkenyl, alkynyl, aryl, arylalkyl, cycloalkyl, carbonyl, ether, haloalkyl, heteroaryl and heterocyclyl.

The term “carbamate” means a group of the form -Z¹OC(O)N(Z¹)—, —Z¹OC(O)N(Z¹)Z¹—, or —OC(O)N(Z¹)₂, where each Z¹ can be, independently, an alkoxy, aryloxy, alkyl, alkenyl, alkynyl, aryl, arylalkyl, cycloalkyl, ether, formyl, haloalkyl, heteroaryl, and heterocyclyl. Carbamates include, e.g., arylcarbamates and heteroaryl carbamates.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “formamide” refers to compounds comprising the —NC(O)H formamide group. Formamides include compounds having the formula HC(O)NZ¹Z² wherein Z¹ and Z² can be, independently, hydrogen or an alkyl, alkenyl, alkynyl, aryl, arylalkyl, cycloalkyl, carbonyl, ether, haloalkyl, heteroaryl and heterocyclyl.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “phosphonium group” refers to a compound of the formula —PR₃⁺, wherein each R is independently hydrogen, alkyl, alkenyl, aryl, and aralkyl, as defined herein. The term “alkyl phosphonium” is a subset of phosphonium, wherein at least one R is an alkyl group. Alkyl phosphonium may be monoalkyl or dialkyl or trialkyl phosphonium. Similarly, the term “aryl phosphonium” is a subset of phosphonium, wherein at least one R is an aryl group. Aryl phosphonium may be monoaryl or diaryl or triaryl phosphonium. In addition, the term “arylalkyl phosphonium” is a subset of phosphonium, wherein the phosphorous atom comprises either at least one aryl group and at least one alkyl group.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Compounds

Disclosed herein are compounds and methods of making and use thereof. For example, disclosed herein are compounds comprising a structure represented by Formula I, or a pharmaceutically acceptable salt thereof:

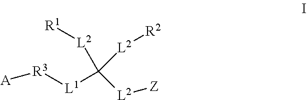

wherein
A comprises a mitochondrial targeting moiety;
Z comprises a lipid chain;
R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene, or —R^{a}X—, wherein R^{a} is a C₀-C₄ alkylene and X is a detectable-group, such as silyl group or fluorophore.

In some examples of Formula I, the mitochondrial targeting moiety comprises a delocalized lipophilic cation. Delocalized lipophilic cations (DLCs) represent a group of compounds capable of penetrating plasma and mitochondrial membranes and accumulate in mitochondria. Suitable examples of delocalized lipophilic cations that penetrate the hydrophobic barriers of plasma and mitochondrial membranes include Rhodamine-123, rhodacyanine MKT-077, dequalinium, triphenylphosphonium, guanidinium cations, and F16. In some examples of Formula I, the mitochondrial targeting moiety comprises an aryl phosphine or aryl phosphonium group (e.g., a monoaryl phosphonium, diaryl phosphonium, or triaryl phosphonium). In some examples of Formula I, the mitochondrial targeting moiety comprises a phosphonium group (e.g., an aryl phosphonium group). In some examples of Formula I, the mitochondrial targeting moiety comprises a substituted or unsubstituted triphenylphosphonium (TPP). In some examples of Formula I, the mitochondrial targeting moiety comprises a mitochondria targeting peptide.

In various examples according to Formula I, Z is a lipid chain. As used herein, the term “lipid chain” and the like, refers to saturated or unsaturated hydrocarbon chains derived from hydrophobic tails of lipids, for example alkyl, alkenyl or alkynyl chains, as described elsewhere herein. The lipid chain may be derived from di-aliphatic chain lipids, phospholipids, diglycerides, di-aliphatic glycolipids, sphingomyelin, glycosphingolipid, steroidal lipids, or hydrophilic polymer derivatized lipids. In some embodiments, the lipid chain comprises 4 to 40 carbons, e.g., 4 to 20 carbons, 10 to 40 carbons, 10 to 30 carbons, 10 to 20 carbons, or 20 to 30 carbons. In some examples of Formula I, Z is a substituted or unsubstituted alkyl. In some examples of Formula I, Z is a substituted or unsubstituted C₅-C₂₅ alkyl. For example, Z in various examples can be a C₁₀-C₂₅ linear alkyl (e.g., an unsubstituted C₁₀-C₂₅ linear alkyl). In some examples of Formula I, Z is octadecyl.

In some examples of Formula I, R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor. Glycolysis inhibitors are generally known in the art and can include small molecules, proteins (e.g., antibodies) or nucleic acids (e.g. siRNA, miRNA, dsRNA). In some examples, the glycolysis inhibitor is a glucose analog. In some examples, the glycolysis inhibitor is a molecule that directly inhibits one or more enzymes in the glycolysis pathway (e.g. hexokinase, phosphofructokinase and/or pyruvate kinase). Suitable glycolysis inhibitors include, for example, lonidamine, dichloroacetate, alpha-tocopheryl succinate, methyl jasmonate, betulinic acid, and resveratrol, A-385358, ABT-263, ABT-737, AT-101, 2-amino-6-bromo-4-(1-cyano-2-ethoxy-2-oxoethyl)-4H-chromene-3-carboxylate (HA 14-1), LDH-A shRNA, orlistat, SB-204990, soraphen A, 4-(N-(s-glutathionylacetate)aminophenylarsenoxide (GSAO), clodronate, PK11 195, menadione, beta-lapachone, CD437, gamitrinibs, 8-(2-chloro-3,4,5-trimethoxybenzyl)-2-fluoro-9-(pent-4-nyl)-9H-purin-6-amine (PU24Fcl), (8-(6-bromobenzo[d][1,3,]dioxyl-5-ylthio)-9-(pent-4-nyl)-9H-purin-6-amine (PUH58), 8-(6-iodobenzo[d][1,3,]dioxyl-5-ylthio)-9-(3-isopropylamino)propyl-9H-purin-6-amine (PUH71), shepherdin, 2-methoxy estradiol, tetrathiomolybdate, buthionine sulphoximine, dimethylamino-parthenolide, parthenolide, imexons, magafodipir, menadione, motexafin gadolinium, PEITCs, elescomol (STA-4783), all trans-retinoic acid, 6-[3-(1-adamantyl)-4-hydroxyphenyl]-2-naphthalene carboxylic acid, E-3-(4′-hydroxy-3′-adamantylbiphenyl-4yl)acrylic acid, 3-bromopyruvate, butyric acid, 2-deoxyD-glucose, arsenite trioxide, or betulinic acid. As used herein, the term “fatty acid inhibitor,” refers to a moiety able to inhibit (e.g., prevent, or at least decrease or inhibit the activity by an order of magnitude or more) a reaction within the fatty acid metabolism pathway, such as an enzyme-catalyzed reaction within the pathway. The inhibitor can inhibit the enzyme, e.g., by binding to the enzyme or otherwise interfering with operation of the enzyme (for example, by blocking an active site or a docking site, altering the configuration of the enzyme, competing with an enzyme substrate for the active site of an enzyme, etc.), and/or by reacting with a coenzyme, cofactor, etc. necessary for the enzyme to react with a substrate. Some non-limiting examples of fatty acid inhibitors include cerulenin, 5-(tetradecyloxy)-2-furoic acid, oxfenicine, methyl palmoxirate, metoprolol, amiodarone, perhexiline, aminocarnitine, hydrazonopropionic acid, 4-bromocrotonic acid, trimetazidine, ranolazine, hypoglycin, dichloroacetate, methylene cyclopropyl acetic acid, and beta-hydroxy butyrate. The term “glutamine oxidation inhibitor,” as used herein refers to moieties that are able to interfere with glutamine metabolism. Some examples of suitable glutamine oxidation inhibitors include (25)-Amino[(55)-3-chloro-4,5-dihydro-1,2-oxazol-5-yljethanoic acid (Acivicin) or 6-Diazo-5-oxo-L-norleucine (DON) and Compound 968 (5-(3-Bromo-4-(dimethylamino)phenyl)-2,2-dimethyl-2,3,5,6-tetrahydrobenzo[a]phenanthridin-4(1H)-one). In some examples of Formula I, R¹ and R² are the same. In some examples of Formula I, R¹ and R² are different. In some examples of Formula I, at least one of R¹ or R² is selected from the group consisting of:

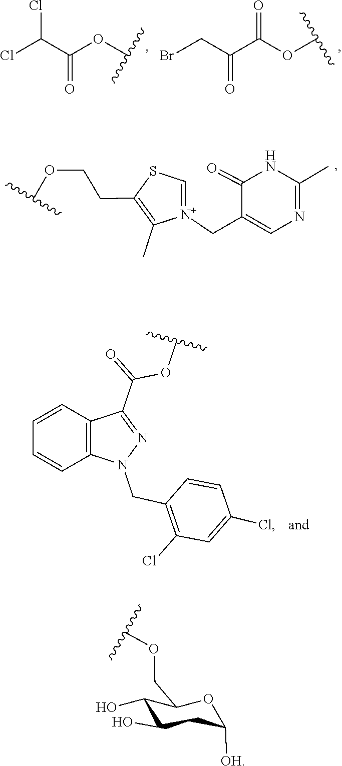

In some examples of Formula I, L¹ is an amide.

In some examples of Formula I, at least one of R¹ or R² is

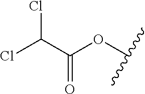 [SMILES: *OC(=O)C(Cl)Cl]

In some examples of Formula I, L¹ is a substituted or unsubstituted alkylene, a carbonyl, an ester, an amide, a carbamate ester, an amine, an ether, a carbonate ester, a thioether, a thioester, or a urea. In some examples of Formula I, L¹ is an amide.

In some examples of Formula I, each L² is independently a direct bond, a C₁-C₄ alkylene, or —R^{a}X—, wherein R^{a} is a C₀-C₄ alkylene and X is a detectable-group. As used herein, the term “detectable group” refers to a chemical moiety, the presence of which can be identified, and the relative quantity and, in certain instances, biodistribution thereof, can be measured using assays as known to those skilled in the art. For example, methods for detecting detectable groups can include gas chromatography (GC), liquid chromatography/mass spectroscopy (LC-MS), gas chromatography/mass spectroscopy (GC-MS), nuclear magnetic resonance (NMR), magnetic resonance imaging (MRI), Fourier Transform InfraRed (FT-IR), and inductively coupled plasma mass spectrometry (ICP-MS). It is further understood that mass spectrometry techniques include, but are not limited to, the use of magnetic-sector and double focusing instruments, transmission quadrapole instruments, quadrupole ion-trap instruments, time-of-flight instruments (TOF), Fourier transform ion cyclotron resonance instruments (FT-MS), and matrix-assisted laser desorption/ionization time-of-flight mass spectrometry (MALDI-TOF MS). In some examples, the detectable-group comprises a chemical moiety capable of being quantified by inductively coupled plasma mass spectrometry (ICP-MS).

In some examples of Formula I, the detectable group is selected from halogen-containing group, a fluorescent group, a radioisotope-containing group, and combinations thereof. In some examples of Formula I, the detectable group is selected from silyl and silyl ether. For example, the silyl ether can be defined by the formula —O—Si R⁵R⁶—, wherein R⁵ and R⁶ are independently hydrogen or a C₁-C₈ alkyl. In some examples of Formula I, R⁵ and R⁶ are the same. In some examples of Formula I, R⁵ and R⁶ are different.

In some examples, the compound comprises Formula II, or a pharmaceutically acceptable salt thereof:

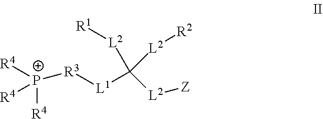

wherein
Z comprises a lipid chain;
R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
each R⁴ is independently a substituted or unsubstituted aryl or heteroaryl;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene, or —R^{a}X—, wherein R^{a} is a C₀-C₄ alkylene and X is a detectable-group.

In various examples according to Formula II, Z is a lipid chain. In some embodiments, the lipid chain comprises 4 to 40 carbons, e.g., 4 to 20 carbons, 10 to 40 carbons, 10 to 30 carbons, 10 to 20 carbons, or 20 to 30 carbons. In some examples of Formula II, Z is a substituted or unsubstituted alkyl. In some examples of Formula II, Z is a substituted or unsubstituted C₅-C₂₅ alkyl. For example, Z can be a C₁₀-C₂₅ linear alkyl (e.g., an unsubstituted C₁₀-C₂₅ linear alkyl). In some examples of Formula II, Z is octadecyl.

In some examples of Formula II, R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor. As stated above, glycolysis inhibitors are generally known in the art and can include small molecules, proteins (e.g., antibodies) or nucleic acids (e.g. siRNA, miRNA, dsRNA). In some examples, the glycolysis inhibitor is a glucose analog. In some examples, the glycolysis inhibitor is a molecule that directly inhibits one or more enzymes in the glycolysis pathway (e.g. hexokinase, phosphofructokinase and/or pyruvate kinase). Exemplary glycolysis inhibitors include, for example, lonidamine, dichloroacetate, alpha-tocopheryl succinate, methyl jasmonate, betulinic acid, and resveratrol, A-385358, ABT-263, ABT-737, AT-101, 2-amino-6-bromo-4-(1-cyano-2-ethoxy-2-oxoethyl)-4H-chromene-3-carboxylate (HA 14-1), LDH-A shRNA, orlistat, SB-204990, soraphen A, 4-(N-(s-glutathionylacetate)aminophenylarsenoxide (GSAO), clodronate, PK11 195, menadione, beta-lapachone, CD437, gamitrinibs, 8-(2-chloro-3,4,5-trimethoxybenzyl)-2-fluoro-9-(pent-4-nyl)-9H-purin-6-amine (PU24Fcl), (8-(6-bromobenzo[d][1,3,]dioxyl-5-ylthio)-9-(pent-4-nyl)-9H-purin-6-amine (PUH58), 8-(6-iodobenzo[d][1,3,]dioxyl-5-ylthio)-9-(3-isopropylamino)propyl-9H-purin-6-amine (PUH71), shepherdin, 2-methoxy estradiol, tetrathiomolybdate, buthionine sulphoximine, dimethylamino-parthenolide, parthenolide, imexons, magafodipir, menadione, motexafin gadolinium, PEITCs, elescomol (STA-4783), all trans-retinoic acid, 6-[3-(1-adamantyl)-4-hydroxyphenyl]-2-naphthalene carboxylic acid, E-3-(4′-hydroxy-3′-adamantylbiphenyl-4yl)acrylic acid, 3-bromopyruvate, butyric acid, 2-deoxyD-glucose, arsenite trioxide, or betulinic acid. In some examples of Formula II, the fatty acid inhibitor is cerulenin, 5-(tetradecyloxy)-2-furoic acid, oxfenicine, methyl palmoxirate, metoprolol, amiodarone, perhexiline, aminocarnitine, hydrazonopropionic acid, 4-bromocrotonic acid, trimetazidine, ranolazine, hypoglycin, dichloroacetate, methylene cyclopropyl acetic acid, or beta-hydroxy butyrate. In some examples of Formula II, the glutamine oxidation inhibitors is (25)-Amino[(55)-3-chloro-4,5-dihydro-1,2-oxazol-5-yl]ethanoic acid (Acivicin) or 6-Diazo-5-oxo-L-norleucine (DON) or Compound 968 (5-(3-Bromo-4-(dimethylamino)phenyl)-2,2-dimethyl-2,3,5,6-tetrahydrobenzo[a]phenanthridin-4(1H)-one). In some examples of Formula II, R¹ and R² are the same. In some examples of Formula II, R¹ and R² are different. In some examples of Formula II, at least one of R¹ or R² is selected from the group consisting of:

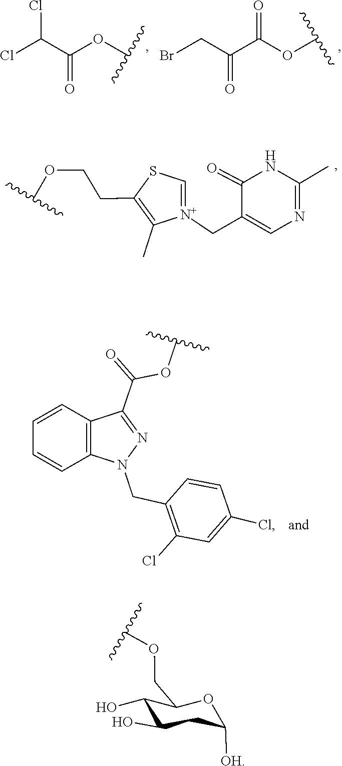

In some examples of Formula II, L¹ is an amide.

In some examples of Formula II, at least one of R¹ or R² is

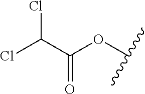 [SMILES: *OC(=O)C(Cl)Cl]

In some examples of Formula II, L¹ is a substituted or unsubstituted alkylene, a carbonyl, an ester, an amide, a carbamate ester, an amine, an ether, a carbonate ester, a thioether, a thioester, or a urea. In some examples of Formula II, L¹ is an amide.

In some examples of Formula II, each L² is independently a direct bond, a C₁-C₄ alkylene, or —R^{a}X—, wherein R^{a} is a C₀-C₄ alkylene and X is a detectable-group.

In some examples of Formula II, the detectable group is selected from halogen-containing group, a fluorescent group, a radioisotope-containing group, and combinations thereof. In some examples of Formula II, the detectable group is selected from silyl and silyl ether. For example, the silyl ether can be defined by the formula —O—Si R⁵R⁶—, wherein R⁵ and R⁶ are independently hydrogen or a C₁-C₈alkyl. In some examples of Formula II, R⁵ and R⁶ are the same. In some examples of Formula II, R⁵ and R⁶ are different.

In some examples, the compound comprises Formula III, or a pharmaceutically acceptable salt thereof:

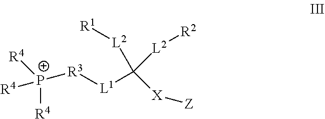

wherein
Z comprises a lipid chain;
R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
each R⁴ is independently a substituted or unsubstituted aryl or heteroaryl;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene, and
X is a detectable-group.

In various examples according to Formula III, Z is a lipid chain. In some embodiments, the lipid chain comprises 4 to 40 carbons, e.g., 4 to 20 carbons, 10 to 40 carbons, 10 to 30 carbons, 10 to 20 carbons, or 20 to 30 carbons. In some examples of Formula III, Z is a substituted or unsubstituted alkyl. In some examples of Formula III, Z is a substituted or unsubstituted C₅-C₂₅ alkyl. For example, Z can be a C₁₀-C₂₅ linear alkyl (e.g., an unsubstituted C₁₀-C₂₅ linear alkyl). In some examples of Formula III, Z is octadecyl.

In some examples of Formula III, R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor. As stated above, glycolysis inhibitors are generally known in the art and can include small molecules, proteins (e.g., antibodies) or nucleic acids (e.g. siRNA, miRNA, dsRNA). In some examples, the glycolysis inhibitor is a glucose analog. In some examples, the glycolysis inhibitor is a molecule that directly inhibits one or more enzymes in the glycolysis pathway (e.g. hexokinase, phosphofructokinase and/or pyruvate kinase). Exemplary glycolysis inhibitors include, for example, lonidamine, dichloroacetate, alpha-tocopheryl succinate, methyl jasmonate, betulinic acid, and resveratrol, A-385358, ABT-263, ABT-737, AT-101, 2-amino-6-bromo-4-(1-cyano-2-ethoxy-2-oxoethyl)-4H-chromene-3-carboxylate (HA 14-1), LDH-A shRNA, orlistat, SB-204990, soraphen A, 4-(N-(s-glutathionylacetate)aminophenylarsenoxide (GSAO), clodronate, PK11 195, menadione, beta-lapachone, CD437, gamitrinibs, 8-(2-chloro-3,4,5-trimethoxybenzyl)-2-fluoro-9-(pent-4-nyl)-9H-purin-6-amine (PU24Fcl), (8-(6-bromobenzo[d][1,3,]dioxyl-5-ylthio)-9-(pent-4-nyl)-9H-purin-6-amine (PUH58), 8-(6-iodobenzo[d][1,3,]dioxyl-5-ylthio)-9-(3-isopropylamino)propyl-9H-purin-6-amine (PUH71), shepherdin, 2-methoxy estradiol, tetrathiomolybdate, buthionine sulphoximine, dimethylamino-parthenolide, parthenolide, imexons, magafodipir, menadione, motexafin gadolinium, PEITCs, elescomol (STA-4783), all trans-retinoic acid, 6-[3-(1-adamantyl)-4-hydroxyphenyl]-2-naphthalene carboxylic acid, E-3-(4′-hydroxy-3′-adamantylbiphenyl-4yl)acrylic acid, 3-bromopyruvate, butyric acid, 2-deoxyD-glucose, arsenite trioxide, or betulinic acid. In some examples of Formula III, the fatty acid inhibitor is cerulenin, 5-(tetradecyloxy)-2-furoic acid, oxfenicine, methyl palmoxirate, metoprolol, amiodarone, perhexiline, aminocarnitine, hydrazonopropionic acid, 4-bromocrotonic acid, trimetazidine, ranolazine, hypoglycin, dichloroacetate, methylene cyclopropyl acetic acid, or beta-hydroxy butyrate. In some examples of Formula III, the glutamine oxidation inhibitors is (25)-Amino[(55)-3-chloro-4,5-dihydro-1,2-oxazol-5-yl]ethanoic acid (Acivicin) or 6-Diazo-5-oxo-L-norleucine (DON) or Compound 968 (5-(3-Bromo-4-(dimethylamino)phenyl)-2,2-dimethyl-2,3,5,6-tetrahydrobenzo[a]phenanthridin-4(1H)-one). In some examples of Formula III, R¹ and R² are the same. In some examples of Formula III, R¹ and R² are different. In some examples of Formula III, at least one of R¹ or R² is selected from the group consisting of:

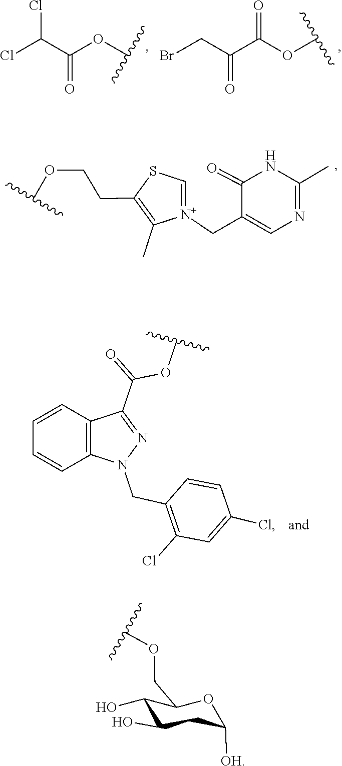

In some examples of Formula III, L¹ is an amide.

In some examples of Formula III, at least one of R¹ or R² is

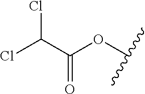 [SMILES: *OC(=O)C(Cl)Cl]

In some examples of Formula III, L¹ is a substituted or unsubstituted alkylene, a carbonyl, an ester, an amide, a carbamate ester, an amine, an ether, a carbonate ester, a thioether, a thioester, or a urea. In some examples of Formula III, L¹ is an amide.

In some examples of Formula III, each L² is independently a direct bond, a C₁-C₄ alkylene, or —R^{a}X—, wherein R^{a} is a C₀-C₄ alkylene and X is a detectable-group.

In some examples of Formula III, the detectable group is selected from halogen-containing group, a fluorescent group, a radioisotope-containing group, and combinations thereof. In some examples of Formula III, the detectable group is selected from silyl and silyl ether. For example, the silyl ether can be defined by the formula —O—Si R⁵R⁶—, wherein R⁵ and R⁶ are independently hydrogen or a C₁-C₈ alkyl.

In some examples of Formula III, R⁵ and R⁶ are the same. In some examples of Formula III, R⁵ and R⁶ are different.

In some examples, the compound comprises Formula IV, or a pharmaceutically acceptable salt thereof:

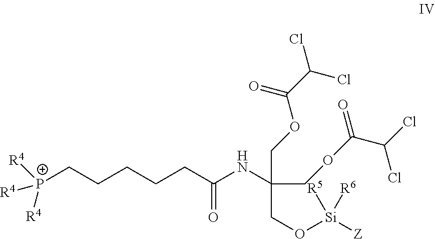

wherein
Z comprises a lipid chain;
each R⁴ is independently a substituted or unsubstituted aryl or heteroaryl; and
R⁵ and R⁶ are independently hydrogen or a C₁-C₈ alkyl.

In various examples according to Formula IV, Z is a lipid chain. In some embodiments, the lipid chain comprises 4 to 40 carbons, e.g., 4 to 20 carbons, 10 to 40 carbons, 10 to 30 carbons, 10 to 20 carbons, or 20 to 30 carbons. In some examples of Formula IV, Z is a substituted or unsubstituted alkyl. In some examples of Formula IV, Z is a substituted or unsubstituted C₅-C₂₅ alkyl. For example, Z can be a C₁₀-C₂₅ linear alkyl (e.g., an unsubstituted C₁₀-C₂₅ linear alkyl). In some examples of Formula IV, Z is octadecyl.

In some examples, the compound comprises:

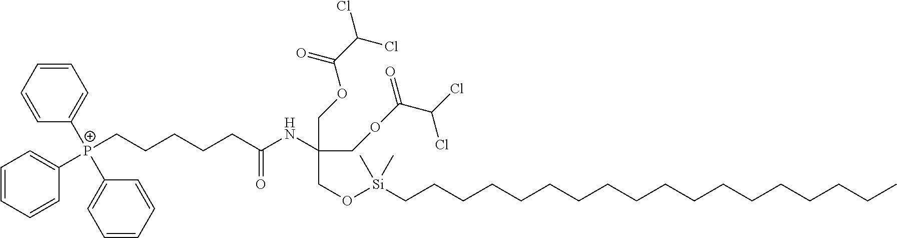 [SMILES: CCCCCCCCCCCCCCCCCC[Si](C)(C)OCC(COC(=O)C(Cl)Cl)(COC(=O)C(Cl)Cl)NC(=O)CCCCC[P+](c1ccccc1)(c1ccccc1)c1ccccc1]

or a pharmaceutically acceptable salt thereof.

### Nanoparticles

Also disclosed herein is a nanoparticle (e.g., one or more nanoparticles) comprising any of the compounds disclosed herein and a polyethylene glycol-lipid.

The nanoparticle can be of any shape. (e.g., a sphere, a rod, a quadrilateral, an ellipse, a triangle, a polygon, etc.). In some examples, the nanoparticle can have a regular shape, an irregular shape, an isotropic shape, an anisotropic shape, or a combination thereof. In some examples, the nanoparticles are substantially spherical in shape.

The nanoparticles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

The nanoparticles can, for example, have an average particle size of 50 nanometers (nm) or more (e.g., 60 nm or more, 70 nm or more, 80 nm or more, 90 nm or more, 100 nm or more, 110 nm or more, 120 nm or more, 130 nm or more, 140 nm or more, 150 nm or more, 160 nm or more, 170 nm or more, 180 nm or more, 190 nm or more, 200 nm or more, 225 nm or more, 250 nm or more, 275 nm or more, 300 nm or more, 325 nm or more, 350 nm or more, 375 nm or more, 400 nm or more, 425 nm or more, 450 nm or more, or 475 nm or more). In some examples, the nanoparticles can have an average particle size of 500 nm or less (e.g., 475 nm or less, 450 nm or less, 425 nm or less, 400 nm or less, 375 nm or less, 350 nm or less, 325 nm or less, 300 nm or less, 275 nm or less, 250 nm or less, 225 nm or less, 200 nm or less, 190 nm or less, 180 nm or less, 170 nm or less, 160 nm or less, 150 nm or less, 140 nm or less, 130 nm or less, 120 nm or less, 110 nm or less, 100 nm or less, 90 nm or less, 80 nm or less, 70 nm or less, or 60 nm or less). The average particle size of the nanoparticles can range from any of the minimum values described above to any of the maximum values described above. For example, the nanoparticles can have an average particle size of from 50 nm to 500 nm (e.g., from 50 nm to 275 nm, from 275 nm to 500 nm, from 50 nm to 200 nm, from 200 nm to 350 nm, from 350 nm to 500 nm, from 60 nm to 500 nm, from 50 nm to 475 nm, from 60 nm to 475 nm, from 100 nm to 200 nm, from 120 nm to 140 nm, or from 150 nm to 200 nm).

With respect to particle size distribution characterization, a parameter used to define the size range of the nanoparticles is called the “polydispersity index” (PDI). The term “polydispersity” (or “dispersity” as recommended by IUPAC) is used to describe the degree of non-uniformity of a size distribution of particles. PDI is basically a representation of the distribution of size populations within a given sample. The numerical value of PDI ranges from 0.0 (for a perfectly uniform sample with respect to the particle size) to 1.0 (for a highly polydisperse sample with multiple particle size populations).

In some examples, the nanoparticles can have a polydispersity index of 0.3 or less (e.g., 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less).

In some examples, the nanoparticles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

In some examples, the polyethylene glycol-lipid comprises a biodegradable polymer. Such polymers are recognizable and identifiable by one or ordinary skill in the art. Non-limiting examples of synthetic, biodegradable polymers include: poly(amides) such as poly(amino acids) and poly(peptides); poly(esters) such as poly(lactic acid), poly(glycolic acid), poly(lactic-co-glycolic acid) (PLGA), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), fibrin, fibrinogen, cellulose, starch, collagen, and hyaluronic acid, copolymers and mixtures thereof. The properties and release profiles of these and other suitable polymers are known or readily identifiable. In some examples, the polyethylene glycol-lipid includes a poly(D,L-lactic-co-glycolic acid)-block-poly(ethylene glycol)-triphenylphosphonium (PLGA-b-PEG-TPP) polymer.

### Pharmaceutical Compositions

Also disclosed herein is a pharmaceutical composition comprising a pharmaceutically acceptable carrier and an effective amount of the compounds or nanoparticles described herein.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

For the treatment of oncological disorders, the compounds or compositions disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds or compositions disclosed herein. For example, the compounds or compositions disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In various examples, composition includes a second compound or composition comprising a therapeutic agent. In some examples, the therapeutic agent comprises an anti-cancer agent.

Also disclosed herein is a method of treating a disease or condition in a subject. In various examples, the method includes: administering to the subject an effective amount of any of the compounds, nanoparticles, or the pharmaceutical composition disclosed herein. In some examples, the disease is HIV. In some examples, the disease is Alzheimer's disease. In some examples, the disease is Parkinson's disease. In some examples, the disease is Amyotrophic Lateral Sclerosis

In some examples, the disease comprises a cancer. Exemplary cancers suitable for the present method include prostate cancer, lung cancer, breast cancer, brain cancer, ovarian cancer, lymphoma cancer, leukemia cancer, head and neck cancer, pancreatic cancer, cervical cancer, colon cancer and rectal cancer, endometrial cancer, esophagus cancer, liver cancer, penile cancer, skin-melanoma, skin-nonmelanoma, stomach cancer, testicular cancer, vaginal cancer, uterine cancer, vulvar cancer, paranasal cancer, oropharyngeal cancer, or laryngeal cancer. In some examples, the cancer is breast cancer.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. All-In-One Pyruvate Dehydrogenase Kinase Inhibitor for Tracking, Targeting, and Enhanced Stability and Efficacy

Most cell types have a standard or preferred metabolic pathway that they utilize to meet their energy requirement to function. In the disease state, however, these cells may switch to exhibit altered metabolism and utilize a typically non-preferred method.¹⁻² One such altered metabolic pathway is glycolysis which is exaggerated in many disease-state cells.³ Even under normoxic conditions which can be regarded as ideal for aerobic mitochondrial respiration, these cells still prefer to utilize glycolysis.⁴ Despite a lesser overall yield of ATP than mitochondrial respiration, glycolysis requires less energy input and can therefore be hijacked to operate at a remarkably higher rate; thus, allowing diseased cells to grow efficiently. There are two aspects of this exaggerated glycolytic dependence in diseased cells that are important to understand. Firstly, glycolysis is completed at a much faster rate and secondly, diseased cells have metabolic flexibility.⁵ Thus, disruption of glycolysis in such disease types could therefore inhibit their proliferation and bring therapeutic efficacy in such diseases.

One of the most studied target enzymes to achieve glycolytic inhibition is pyruvate dehydrogenase kinase-1 (PDK1).⁶⁻⁸ This enzyme is a major inhibitory regulator of the enzyme pyruvate dehydrogenase (PDH), which is essential for the oxidative decarboxylation of glycolytic pyruvate to form acetyl-CoA for mitochondrial respiration. By targeting and inhibiting PDK1, PDH can operate in an unregulated manner, as the negative feedback inhibition loop created by acetyl-CoA is now lost. Dichloroacetate (DCA) which was initially thought of as a PDK1 inhibitor generated significant interest for its glycolysis inhibitory properties in the context of lactic acidosis which was later extended to utilize in tumor metabolism.⁸⁻¹⁴ Initial studies resulted in significant interest which unveiled additional mode of action and targets for DCA, a synergistic combination of this molecule with other forms of cancer therapies. Recent studies have developed several molecular entities containing DCA to utilize the functional activity of this kinase inhibitor and have also documented DCA's glycolysis inhibitor action resulting in beneficial regulation of the immunosuppressive nature of cancers.¹⁵ But due to the lack of effective cellular uptake of DCA and poor localization inside the mitochondrial matrix where the PDK1 is located, the therapeutic applications of DCA are greatly limited due to high DCA doses which are required for tumor growth suppression¹⁶ and associated neurological toxicity. In order to introduce physiologically relevant DCA doses and to engineer the anionic form of DCA to partition across the inner mitochondrial membrane (IMM) to access PDK1, the study developed a DCA-based prodrug, Mito-DCA,⁸ which has three DCA groups and a triphenylphosphonium (TPP) cation to utilize mitochondrial membrane potential (Δψₘ) to partition into the mitochondrial matrix of cells resulting substantially lower minimum effective dosage.

### Materials and Methods

Materials. Description of materials and methods, chemicals, biochemical, and other experimental and characterization methods are described in Supplementary Information.

Synthesis of compounds. Methoxy (octadecyldimethyl)silane was synthesized according to literature report.¹⁷ Mito-DCA was synthesized according to a previous reported method.⁸ TPP-(CH₂)₅—COOH) and biodegradable polymer PLGA-b-PEG₃₃₅₀-TPP, which were used for nanoparticle synthesis, were synthesized according to a previous report.¹⁸

Cell lines and cell culture. Breast cancer cell line 4T1 was obtained from American Type Culture Collection (ATCC). The cells were cultured at 37° C. in 5% CO₂ in Roswell Park Memorial Institute Medium (RPMI) 1640 supplemented with 10% fetal bovine serum (FBS), 1% penicillin/streptomycin, 1% HEPES and 1% sodium pyruvate. Cells were passaged once the flask becomes 80% confluent. The breast cancer brain metastasis MDA-MB-231BR cells were procured from Dr. Joan Massagué, Antibody and Bioresource Core Facility at Memorial Sloan Kettering Cancer Center. These cells were grown in Dulbecco's Modified Eagle's Medium (DMEM) supplemented with an additional 10% FBS, 1% each of penicillin/streptomycin, L-glutamine, and sodium pyruvate. The cells were cultured at 37° C. in 5% CO₂. Similar passaging was followed for both the cell lines.

Animals. BALB/c nude female mice (4-5 weeks old) were purchased from Jackson Laboratory. All animals were handled in accordance with “The Guide for the Care and Use of Laboratory Animals” of American Association for Accreditation of Laboratory Animal Care (AAALAC), Animal Welfare Act (AWA), and other applicable federal and state guidelines. All animal work presented here was approved by Institutional Animal Care and Use Committee (IACUC) of University of Miami (UM) Miller School of Medicine (UM Animal Welfare Assurance No. A3224-01, Animal Protocol Number: 20-171, IBC Protocol Number: IBC 20-140). All housing, surgical procedures, and experimental protocols were approved by the IACUC Committee of UM. Animals had free access to diet and water during all experiments.

### Results And Discussion

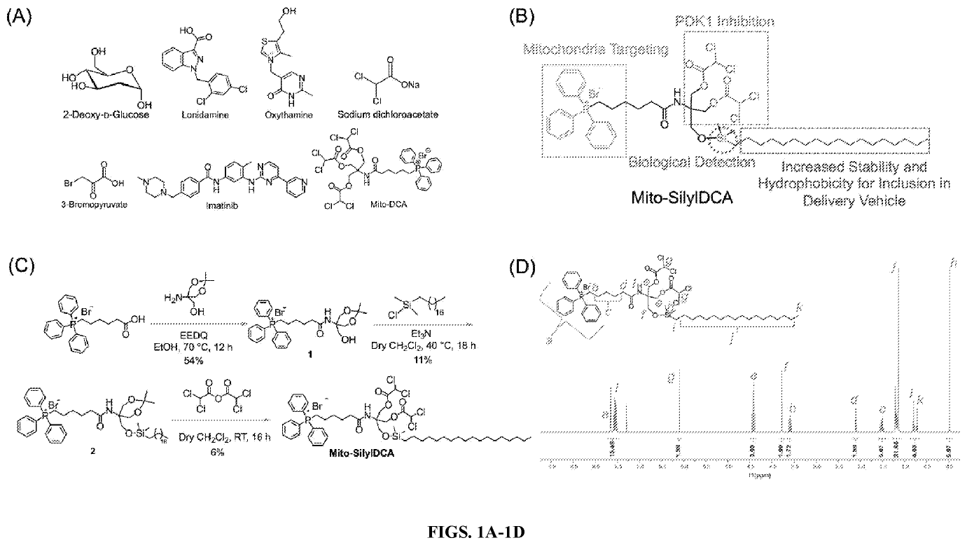

Mito-SilylDCA, an all-in-one glycolytic inhibitor. Mito-DCA has the ability to selectively alter metabolism in cancer cells by inhibiting glycolysis and overall ATP production to reduce cancer cell proliferation.⁸ However, Mito-DCA suffers from susceptibility to hydrolysis and difficulties in quantification. This series of disadvantages are shared by most of the commercially available glycolytic inhibitors in the market which include DCA, 2-deoxy-D-glucose (2-DG), lonidamine, oxythamine, and imatinib (FIG. 1A). None of these inhibitors can be tracked in biological systems; these molecules cannot be quantified by any direct method to follow their biodistribution (bioD) and pharmacokinetic (PK) parameters. Keeping the landscape of available glycolytic inhibitors in terms of advantages and disadvantages in mind and the benefits of selective glycolytic inhibition of cancer cells could offer when executed in a sophisticated manner, the experiment designed and constructed a new molecule, Mito-SilylDCA (FIG. 1B). In this construct, the lipophilic-TPP cation is used for targeting mitochondria of respirating cells, one of the DCA groups in Mito-DCA was replaced with dimethyl-octadecyl silane moiety to serve as a hydrophobic long silane chain to increase the stability by protecting from hydrolysis under physiological environment, and the presence of silicon (Si) in Mito-SilylDCA molecule can be used to track the molecule in biological samples utilizing quantitative inductively coupled plasma mass spectrometry (ICP-MS) (FIG. 1B). Further, incorporation of long hydrophobic chain in Mito-SilylDCA molecule provided the hydrophobicity to adapt the molecule for its effective incorporation inside a polymeric delivery vehicle composed of biodegradable poly-(lactic-co-glycolic acid) (PLGA)-block (b)-polyethylene glycol (PEG)-TPP polymer to result in T-Mito-SilylDCA-NP for tumor targeted delivery utilizing hyperpolarized mitochondrial network of cancer cells. Benefiting from the introduction of Si in the Mito-SilylDCA, the quantifiable mitochondrial targeted NPs were successfully used to investigate the biodistribution in different organs in vivo. A synthetic route to Mito-SilylDCA is depicted in FIG. 1C. The formation of the precursors for Mito-SilylDCA, i.e., compounds 1 and 2 was confirmed by both NMR and mass spectrometry. The formation of the Mito-SilylDCA was confirmed with ¹H (FIG. 1D), ¹³C, and ³¹P NMR spectroscopy and mass spectrometry. The presence of silicon made the compound become quantifiable using the technique ICP-MS possible to calculate the concentration of Mito-SilylDCA or DCA in various complicated biological environments to better understand the property and activity of the drug.

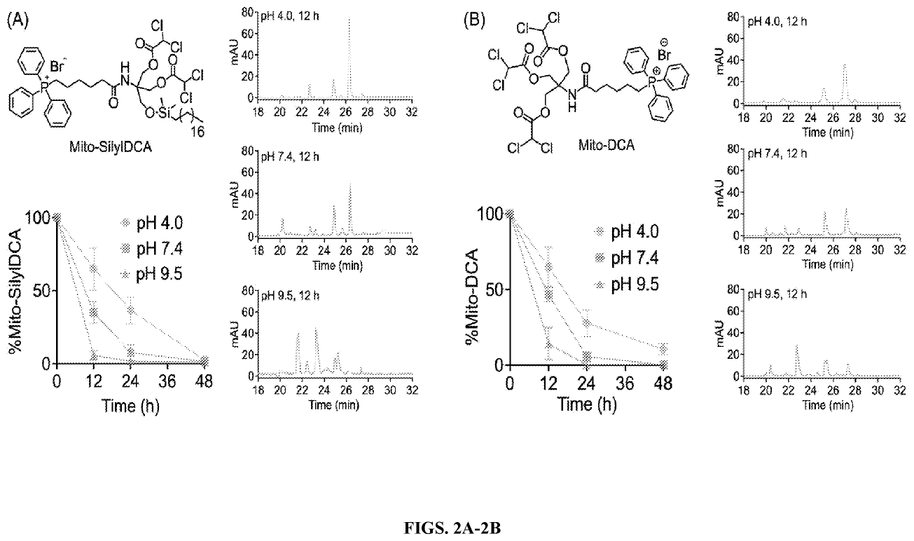

Stability of Mito-SilylDCA. A combination of HPLC and LC-MS analytical techniques were employed to understand the stability of Mito-SilylDCA in different physiological environments. A solution of Mito-SilylDCA or Mito-DCA in DMF-water (2/1, v/v) was incubated for 12, 24, and 48 h at different pH environments of 4.0, 7.4, and 9.5 at room temperature and the degradation behavior of the compounds was analyzed with HPLC and LC-MS (FIGS. 2A and 2B). It was found that, in neutral conditions, both Mito-DCA and Mito-SilylDCA were hydrolyzed in a short time. After 12 h, more than 60% of compounds are decomposed at pH 7.4, which should be due to the hydrolysis of DCA and the silyl chain. Shifting the pH to a more acidic level resulted in a relatively slow degradation, illustrating better stability in an acidic environment. Both Mito-DCA and Mito-SilylDCA were unstable in alkaline conditions and the compounds decomposed easily. A comparison of degradation kinetics for a period of 24 h indicated that decomposition of Mito-SilylDCA was slower than Mito-DCA under acidic conditions, showing that the introduction of silyl chain can prevent the hydrolysis of DCA to some extent by providing a hydrophobic microenvironment to DCA moiety (FIGS. 2A and 2B). The better stability of Mito-SilylDCA compared to Mito-DCA would be beneficial for the long-term storage and further application in biological environment.

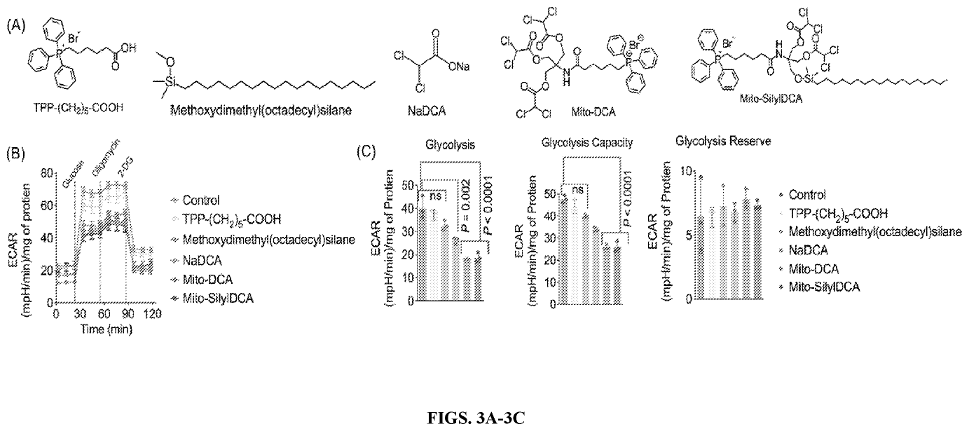

Cellular glycolysis inhibition by Mito-SilylDCA. Glycolysis inhibitory efficacy of Mito-Silyl-DCA was investigated in 4T1 breast cancer cell line which is known to be aggressive and highly glycolytic in nature with an overexpression of PDK1.⁶ The Seahorse glycostress assay was performed to understand the metabolic fluxes of glycolysis. The 4T1 cells grown in normoxia were treated with TPP-(CH₂)₅—COOH) (200 μM), methoxyl(octadecyldimethyl)silane (200 μM), NaDCA (600 μM), Mito-DCA (200 μM), or Mito-Silyl-DCA (200 μM) for 6 h followed by washing and returning the cells to a glucose-free medium (FIG. 3A). The extracellular acidification rate (ECAR) traces were then monitored in response to the sequential administration of glucose, oligomycin, and 2-deoxy-D-glucose (2-DG) to assess glycolysis, glycolytic capacity, and glycolytic reserve, respectively. Mito-DCA and Mito-SilylDCA both showed significant glycolysis inhibition in 4T1 cells from the results. No detectable changes in glycolysis levels were observed for TPP-(CH₂)₅—COOH) and methoxyl(octadecyldimethyl)silane compared with control group (FIG. 3B-3C). The glycolytic inhibitory properties of Mito-SilylDCA and Mito-DCA were found to be significantly higher compered to NaDCA (FIGS. 3B-3C). The data shown here are average from three independent biological replicates. It is important to note that although in Mito-SilylDCA, the number of DCA molecule is less compared to Mito-DCA, the glycolysis inhibition capability was not significantly decreased.

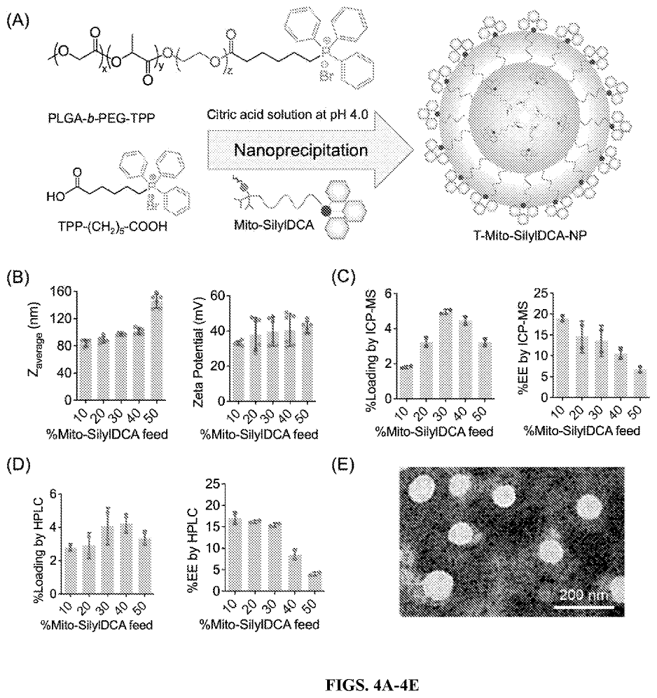

Clinically relevant nanoformulation of Mito-SilylDCA. High accumulation in the tumor region and effective cellular uptake of DCA is advantageous for the improvement of therapeutic efficacy. However, the aqueous solubility of Mito-SilylDCA might obstruct drug transportation and cellular uptake in vivo. To improve the stability and efficiency of Mito-SilylDCA delivery into the mitochondria of cancer cells, the study encapsulated Mito-Silyl-DCA into a biodegradable nanoparticle (NP), which was constructed from PLGA-b-PEG-TPP^{6, 19-20} to result T-Mito-SilylDCA-NPs (FIG. 4A). This self-assembled NPs were constructed using a nanoprecipitation method. The stability studies indicated that Mito-SilylDCA was most stable at an acidic pH. Thus, the experiment performed the nanoprecipitation in citric acid solution of pH 4.0 (FIG. 4A). At this pH, the degradation was relatively slow and Mito-SilylDCA was able to preserve its physicochemical properties while leading to an efficient loading and encapsulation efficiency.⁶ The functionalization with a terminal TPP cation can create a delocalized, lipophilic, positively charged surface on the At this pH, the degradation was relatively slow and Mito-SilylDCA was able to preserve its physicochemical properties while leading to an efficient loading and encapsulation efficiency.⁶ The functionalization with a terminal TPP cation can create a delocalized, lipophilic, positively charged surface on the nanoparticles with properties to target Δψₘ which subsists across the double membranes of respirating mitochondria thus promoting NP association with hyperpolarized mitochondria of cancer cells. To obtain the NPs with highest delivery efficiency, % feed of Mito-SilylDCA was varied from 10% to 50% to create a library of NPs. The properties of T-Mito-SilylDCA-NPs were determined with dynamic light scattering (DLS) for hydrodynamic diameter and surface charge (FIG. 4A). An increase in the size of T-Mito-SilylDCA-NPs was witnessed as the percent feed of Mito-SilylDCA increased. The size of NPs was increased from ~80 nm to ~140 nm, which might be related to the hydrophobicity provided by Mito-SilylDCA. Zeta potential values of these NPs were ~35 mV which would be helpful for the cellular and mitochondrial uptake. The concentration of Mito-SilylDCA in the NPs was determined using HPLC for DCA component and by Si-based ICP-MS benefiting from the introduction of silicon in the molecular structure. By this way, the loading percentage (% loading) and percent encapsulation efficiency (% EE) were calculated (FIGS. 4B and 4C). With the increase of Mito-Silyl-DCA feed, both % loading and % EE initially increased and then declined. A 30% percent Mito-SilylDCA feed was found to have the highest loading and % EE. Thus, this 30% feed NPs were used for all future studies. Percent loading and % EE determined by HPLC, and ICP-MS were in close agreement (FIGS. 4B and 4C).

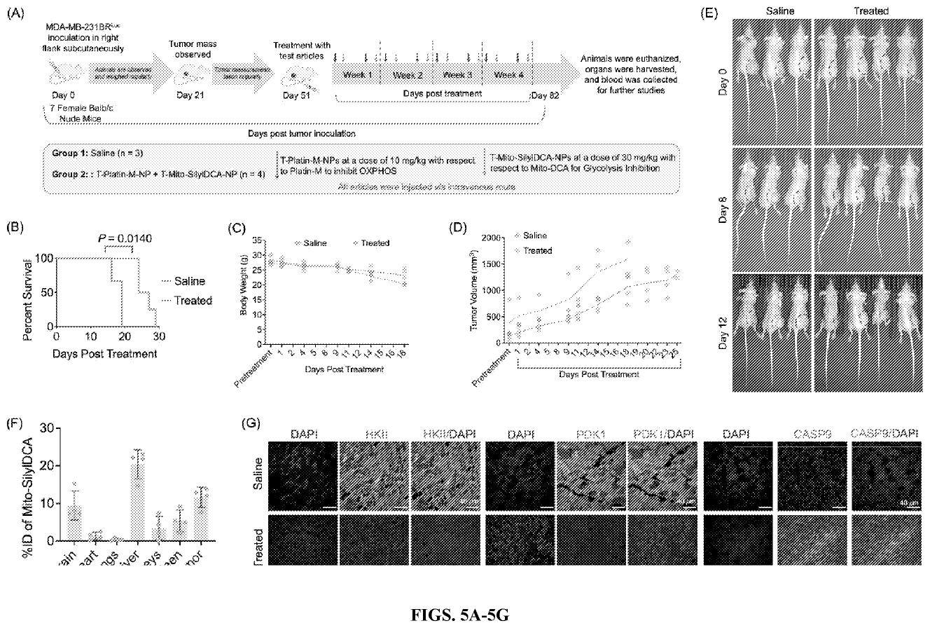

In vivo efficacy of T-Mito-SilylDCA-NPs in triple negative breast cancer xenograft model. The triumph over the triple-negative breast cancer (TNBC) is still far from the realty. This subtype of breast cancer demonstrates the worst prognosis due to the heterogeneity of the diseases, inability to develop targeted therapy, underlying metabolic changes. A combination of standard chemotherapeutics remains as the backbone of treatment regimens of TNBC. Understanding and studying unique changes to metabolic patterns of TNBC and tackling such changes with a combination of therapeutics that work on complementary metabolic pathways will provide innovative therapeutic approaches. Thus, the experiment investigated the efficacy of T-Mito-SilylDCA-NPs in an in vivo mouse model in combination with a cisplatin prodrug, Platin-M²⁰⁻²¹. In earlier experiments, it was discovered that this prodrug can inhibit mitochondrial OXPHOS and simultaneously perform chemotherapeutic actions by forming repair resistant Pt-DNA adduct with mitochondrial DNA (mtDNA) lacking nucleotide excision repair (NER) machinery.²⁰ Without wishing to be bound by theory, the study hypothesized that when animals carrying a tumor model of mitochondrial OXPHOS predominating tumor cells such as TNBC MDA-MB-231BR with brain metastatic potential is used, Platin-M activity will inhibit such OXPHOS resulting an upregulation of glycolysis. Thus, assessment of efficacy of T-Mito-SilylDCA-NPs in such a model can recapitulate unique metabolic changes which happen in aggressive cancers such as TNBC in response to standard treatments. Thus for this study, luciferase tagged MDA-MB-231BR^{Luc} cells in Balb/c nude mice were used. The experiment implanted MDA-MB-231BR^{Luc} cells in the right flank of N=7 female Balb/c nude mice (FIG. 5A). Studies have illustrated that the MDA-MB-231BR^{Luc} cells have an altered metabolic profile.²² They are known to depend upon OXPHOS for their growth and proliferation. Once the tumors were established, these animals were treated with a combination of T-Platin-M-NP and T-Mito-SilylDCA-NP in a sequential manner to inhibit first OXPHOS with T-Platin-M-NP²⁰ and simultaneous inhibition of resulting glycolysis T-Mito-SilylDCA-NP. This study included two groups: Group 1, saline with n=3 animals and group 2, combination of T-Platin-M-NPs and T-Mito-SilylDCA-NPs with n=4 animals (FIG. 5A). The tumor bearing animals were treated with saline or combination therapeutics for 4 weeks following a biweekly treatment regimen which involved tail i.v injections of 10 mg/kg dose of T-Platin-M-NP with respect to Platin-M 24 h prior to 30 mg/kg dose of T-Mito-Silyl-DCA-NP with respect to Mito-SilylDCA. This exploratory study revealed that the combinatorial treatment was able to significantly improve the survival of the animals (FIG. 5B) with minimum to no changes in body weight (FIG. 5C) and reduced tumor proliferation (FIG. 5D). The brightfield image panel in FIG. 5E shows the trend in tumor growth between the saline and combination therapeutic NP treated groups. All the images were taken at the same aperture and field of view was kept at 22.4 cm. The study further performed biodistribution studies to investigate the accumulation of the NPs at the tumor. The data suggested that a significant portion of Mito-SilylDCA accumulated in the tumor when delivered with the NPs (FIG. 5F). Notably, the quantification Mito-SilylDCA in the tumor was possible largely because of the presence of —Si in this molecule, which can be used in mass spectrometry to be able to detect in vivo. The tumor tissues were analyzed using immunofluorescence studies for understanding glycolysis inhibitory properties Mito-SilylDCA in vivo and the consequence on tumor proliferation and growth (FIG. 5G). The experiment observed a downregulation in the expression of HKII and PDK1 in the treated animals, whereas a concomitant upregulation in the Caspase 9 protein expression. These data evidenced efficacious glycolysis inhibitory abilities if Mito-SilylDCA at the tumor and resulting apoptosis inducing property when Mito-SilylDCA is combined with a chemotherapeutic (FIG. 5G).

### SUMMARY

Presently, there are no glycolytic inhibitors with trackability in biological system, extended stability, and physicochemical properties to be incorporated inside a hydrophobic cargo with high efficacy. In this example, the study demonstrated the utility of unique molecule to achieve the above-mentioned properties. A multitude of diseases utilize glycolysis as their rapid source of energy, making these diseases hard to cure. Cancer is one such disease, where the uncontrolled cell proliferation is aided by the glycolytic metabolism. The study revealed that Mito-SilylDCA was able to induce glycolysis inhibition alone or in a nanoparticle. The therapeutic ability of Mito-SilylDCA was further evidenced utilizing an aggressive TNBC mouse model. Thus, it is evident from this study that Mito-SilylDCA alone or in combination with other therapeutics has the potential to be translated into therapeutics targeting glycolysis.

### Example 2. Mito-SilylDCA Molecule and Nanoparticle for Inhibiting Glycolysis in the Brain Viral Reservoirs

HIV infected cells often utilize high glycolysis by promoting GLUT1 and HKI to support viral infection and propagation. Thus, upregulated glycolysis in host cells leads to increased viral infection and amplification. Suboptimal inhibition of glycolysis may be a way to control HIV infection and viral reservoirs (1). One of the most efficient ways to inhibit glycolysis is to block pyruvate dehydrogenase kinase-1 (PDK1). This enzyme is a major inhibitory regulator of the enzyme pyruvate dehydrogenase (PDH), which is essential for the oxidative decarboxylation of glycolytic pyruvate to form acetyl-CoA for mitochondrial respiration. Dichloroacetate (DCA), a small molecule mitochondrial kinase inhibitor, has been identified to have the potential to show such characteristics for cancer chemotherapy (2-5). By utilizing the metabolic switch, DCA reverses cancer cell abnormal metabolism from aerobic glycolysis to glucose oxidation by reducing the activity of mitochondrial PDK1 (6-8), resulting in the negative regulation of PDH and promotion of oxidative phosphorylation (OXPHOS) (4). But due to the lack of effective cellular uptake of DCA and poor localization inside the mitochondrial of tumor cells, the therapeutically applications of DCA are greatly limited and high DCA doses are required for tumor growth suppression (9). In order to introduce physiologically relevant DCA doses into cells and engineer the anionic form of DCA to partition across the inner mitochondrial membrane (IMM) to access PDK1, a DCA-based prodrug named Mito-DCA was developed (7, 10), which has three DCA groups and a triphenylphosphonium (TPP) cation to target the mitochondria of cells and substantially lower minimum effective dosage. However, Mito-DCA suffers from susceptibility to hydrolysis and difficulties in quantification, which is not conductive to its clinical application. This study includes a new molecule, Mito-SilylDCA to have all in one property such as stability, tracking ability, and inclusion in this nanoparticle for brain delivery (FIG. 1A). In this new molecule, dimethyl-octadecyl silane moiety was included as the hydrophobic long silane chain, which could greatly increase the stability of Mito-SilylDCA by protecting from hydrolyzing in physiological environment. Besides, the presence of silicon in the Mito-SilylDCA molecule make it quantifiable under various conditions in the biological samples. To improve the effective delivery and accumulation in the brain to tackle HIV reservoirs, the Mito-SilylDCA was encapsulated in the PLGA-b-PEG-TPP NP to result T-Mito-SilylDCA-NPs. Different Mito-SilylDCA feed percentages were investigated to get the best delivery efficiency. Benefiting from the introduction of silicon in the Mito-SilylDCA molecule, the quantifiable mitochondrial targeted NPs were successfully used to investigate the biodistribution in different organs in vivo.

### Result and Discussion

Design and construction of Mito-SilylDCA. In order to improve the stability and quantification possibility of the glycolysis inhibition prodrug, dimethyl-octadecyl silane moiety was introduced into the molecular structure. Further the presence of silicon made the compound became quantifiable with ICP-MS, which made it possible to calculate the concentration of Mito-SilylDCA or DCA in various complicated biological environments to better understand the property and activity of the drug. To construct Mito-SilylDCA, TPP-Protected Tris (compound 1) was synthesized by reacting (5-carboxypentyl)triphenylphosphonium bromide with protected tris(hydroxymethyl)aminomethane (Tris) in the presence of N-ethoxycarbonyl-2-ethoxy-1,2-dihydroquinoline (EEDQ) to selectively couple amine group with carboxyl acid in the presence of hydroxy functionality. The hydroxyl group of the protected Tris were coupled with chloro(dimethyl)octadecylsilane to improve the stability of the compounds. Finally, the protected hydroxyl group was de-protected under the acidic conditions and coupled with DCA-anhydride to give the final target compound Mito-SilylDCA. The formation of the Mito-SilylDCA compound was confirmed with NMR and LC-MS spectroscopy.

Stability of Mito-SilylDCA. With Mito-SilylDCA in hand, the chemical stability was checked to ensure the efficacy of drug in different physiological environments. A mixed solution of Mito-SilylDCA in DMF/water (1/1, v/v) was incubated for 24 h in different pH environment (4.5, 5.5, 6.5, 7.4, 8.5) at room temperature. Then the solutions were measured with HPLC and LC-MS to check the degradation behavior of the compounds. Mito-SilylDCA was found to be stable in neural and weak acidic conditions.

Glycolysis inhibition in highly glycolytic cells. This example documented inhibition of glycolysis in 4T1 cells by Mito-SilylDCA using Seahorse glycostress assay (FIG. 3B). The Seahorse glycostress assay was performed to check the metabolic fluxes of glycolysis. 4T1 cells were treated with TPP-hexanoic acid (200 μM), methoxyl(octadecyldimethyl)silane (200 μM), sodium dichloroacetate (DCA) (600 μM), Mito-DCA (200 μM), or Mito-SilylDCA (200 μM) for 6 h followed by washout and returned to glycose-free medium. The extracellular acidification rate (ECAR) was monitored in response to the sequential administration of glucose, oligomycin, and 2-deoxy-D-glucose (2-DG) to assess glycolysis, glycolytic capacity, and glycolytic reserve, respectively (FIG. 3C). The results indicated significant inhibition of glycolysis by Mito-SilylDCA in these glycolytic cells.

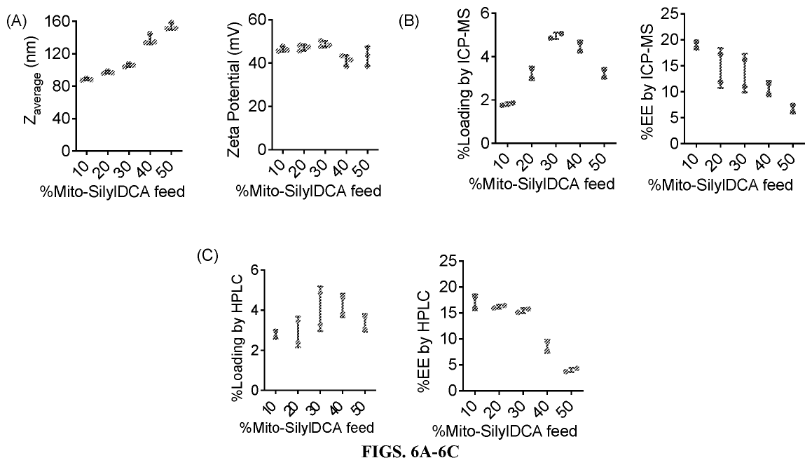

Encapsulation of Mito-SilylDCA in NPs. To improve the stability and efficiency of Mito-SilylDCA delivery in the brain, Mito-SilylDCA was encapsulated in PLGA-PEG-TPP NPs to result T-Mito-SilylDCA-NPs. In order to obtain the nanoparticle with highest deliver efficiency, different feed percentages of Mito-SilylDCA, from 10% to 50%, were attempted to encapsulate in the nanoparticles. The properties of T-Mito-SilylDCA-NPs were determined with dynamic light scattering (DLS) for size and surface charge (FIG. 6A). An increase in the size of T-Mito-SilylDCA-NPs with the percentage feed of Mito-SilylDCA was observed. The size of nanoparticles was increased from ~80 nm to ~140 nm, which might be relevant to the hydrophobic property of Mito-SilylDCA compounds. The zeta potentials were at ~35 mV for all the nanoparticles, which would be helpful for the cellular and mitochondrial uptake. The experiment was able to quantify the loading of Mito-SilylDCA in the NPs both by using HPLC and by ICP-MS benefiting from the introduction of silicon in the molecular structure (FIGS. 6B-6C). With the increase of Mito-SilylDCA feed percentage, the loading percentage and encapsulation efficiency both presented first increase and then decrease trend. The feed of 30% Mito-SilylDCA was found to have highest loading percentage and encapsulation efficiency, which was used for further biodistribution experiment.

Distribution of T-Mito-SilylDCA in NPs in the brain. The present study administered T-Mito-SilylDCA to mice via intravenous injection and quantified the accumulation of NPs using ICP-MS analyses. The quantification of Si was determined throughout various organs which indicated significant accumulation of this compound in the brain (FIG. 5F).

The methods and compositions of the appended claims are not limited in scope by the specific methods and compositions described herein, which are intended as illustrations of a few aspects of the claims and any methods and compositions that are functionally equivalent are within the scope of this disclosure. Various modifications of the methods and compositions in addition to those shown and described herein are intended to fall within the scope of the appended claims. Further, while only certain representative methods, compositions, and aspects of these methods and compositions are specifically described, other methods and compositions and combinations of various features of the methods and compositions are intended to fall within the scope of the appended claims, even if not specifically recited. Thus, a combination of steps, elements, components, or constituents can be explicitly mentioned herein; however, all other combinations of steps, elements, components, and constituents are included, even though not explicitly stated.

### REFERENCES

### Example 1

1. Hanahan, D.; Weinberg, R. A., The hallmarks of cancer. Cell 2000, 100 (1), 57-70.
2. Pavlova, N. N.; Thompson, C. B., The Emerging Hallmarks of Cancer Metabolism. Cell Metab. 2016, 23 (1), 27-47.
3. DeBerardinis, R. J.; Thompson, C. B., Cellular metabolism and disease:
   what do metabolic outliers teach us? Cell 2012, 148 (6), 1132-1144.
4. Kim, J.-w.; Dang, C. V., Cancer's molecular sweet tooth and the Warburg effect. Cancer Res. 2006, 66 (18), 8927-8930.
5. Garber, K., Oncology's energetic pipeline: surging interest in cancer bioenergetics has brought drug developers into the fray, but the field awaits a clinical success. Ken Garber explores the extent to which the concept is entering the mainstream. Nat. Biotechnol. 2010, 28 (9), 888-892.
6. Kolb, D.; Kolishetti, N.; Surnar, B.; Sarkar, S.; Guin, S.; Shah, A. S.; Dhar, S., Metabolic Modulation of the Tumor Microenvironment Leads to Multiple Checkpoint Inhibition and Immune Cell Infiltration. ACS Nano 2020, 14 (9), 11055-11066.
7. Fujiwara, S.; Kawano, Y.; Yuki, H.; Okuno, Y.; Nosaka, K.; Mitsuya, H.; Hata, H., PDK1 inhibition is a novel therapeutic target in multiple myeloma. Br J Cancer 2013, 108 (1), 170-178.
8. Pathak, R. K.; Marrache, S.; Harn, D. A.; Dhar, S., Mito-DCA: A Mitochondria Targeted Molecular Scaffold for Efficacious Delivery of Metabolic Modulator Dichloroacetate. ACS Chem. Biol. 2014, 9 (5), 1178-1187.
9. Bonnet, S.; Archer, S. L.; Allalunis-Turner, J.; Haromy, A.; Beaulieu, C.; Thompson, R.; Lee, C. T.; Lopaschuk, G. D.; Puttagunta, L.; Bonnet, S.; Harry, G.; Hashimoto, K.; Porter, C. J.; Andrade, M. A.; Thebaud, B.; Michelakis, E. D., A mitochondria-K+ channel axis is suppressed in cancer and its normalization promotes apoptosis and inhibits cancer growth. Cancer Cell 2007, 11 (1), 37-51.
10. Dhar, S.; Lippard, S. J., Mitaplatin, a potent fusion of cisplatin and the orphan drug dichloroacetate. Proc. Natl. Acad. Sci. U.S.A. 2009, 106 (52), 22199-22204.
11. Pearson, H., Cancer patients opt for unapproved drug. Nature 2007, 446 (7135), 474-475.
12. Sun, R. C.; Fadia, M.; Dahlstrom, J. E.; Parish, C. R.; Board, P. G.; Blackburn, A. C., Reversal of the glycolytic phenotype by dichloroacetate inhibits metastatic breast cancer cell growth in vitro and in vivo. Breast Cancer Res. Treat. 2010, 120 (1), 253-260.
13. Christofk, H. R.; Vander Heiden, M. G.; Harris, M. H.; Ramanathan, A.; Gerszten, R. E.; Wei, R.; Fleming, M. D.; Schreiber, S. L.; Cantley, L. C., The M2 splice isoform of pyruvate kinase is important for cancer metabolism and tumour growth. Nature 2008, 452 (7184), 230-U74.
14. Kato, M.; Li, J.; Chuang, J. L.; Chuang, D. T., Distinct structural mechanisms for inhibition of pyruvate dehydrogenase kinase isoforms by AZD7545, dichloroacetate, and radicicol. Structure 2007, 15 (8), 992-1004.
15. Babu, E.; Ramachandran, S.; CoothanKandaswamy, V.; Elangovan, S.; Prasad, P. D.; Ganapathy, V.; Thangaraju, M., Role of SLC5A8, a plasma membrane transporter and a tumor suppressor, in the antitumor activity of dichloroacetate. Oncogene 2011, 30 (38), 4026-4037.
16. Stockwin, L. H.; Yu, S. X.; Borgel, S.; Hancock, C.; Wolfe, T. L.; Phillips, L. R.; Hollingshead, M. G.; Newton, D. L., Sodium dichloroacetate selectively targets cells with defects in the mitochondrial ETC. Int. J. Cancer 2010, 127 (11), 2510-2519.
17. Chao, C. G.; Leibham, A. M.; Bergbreiter, D. E., Hydrocarbon Soluble Recyclable Silylation Reagents and Purification Auxiliaries. Org. Lett. 2016, 18 (5), 1214-1216.
18. Surnar, B.; Basu, U.; Banik, B.; Ahmad, A.; Marples, B.; Kolishetti, N.; Dhar, S., Nanotechnology-mediated crossing of two impermeable membranes to modulate the stars of the neurovascular unit for neuroprotection. Proc. Natl. Acad. Sci. U.S.A. 2018, 115 (52), E12333-E12342.
19. Marrache, S.; Dhar, S., Engineering of blended nanoparticle platform for delivery of mitochondria-acting therapeutics. Proc. Natl. Acad. Sci. U.S.A. 2012, 109 (40), 16288-16293.
20. Marrache, S.; Pathak, R. K.; Dhar, S., Detouring of cisplatin to access mitochondrial genome for overcoming resistance. Proc. Natl. Acad. Sci. U.S.A. 2014, 111 (29), 10444-10449.
21. Feldhaeusser, B.; Platt, S. R.; Marrache, S.; Kolishetti, N.; Pathak, R. K.; Montgomery, D. J.; Reno, L. R.; Howerth, E.; Dhar, S., Evaluation of nanoparticle delivered cisplatin in beagles. Nanoscale 2015, 7 (33), 13822-30.
22. Lanning, N. J.; Castle, J. P.; Singh, S. J.; Leon, A. N.; Tovar, E. A.; Sanghera, A.; MacKeigan, J. P.; Filipp, F. V.; Graveel, C. R., Metabolic profiling of triple-negative breast cancer cells reveals metabolic vulnerabilities. Cancer Metab. 2017, 5, 6.

### Example 2

1. J. C. Valle-Casuso et al., Cellular Metabolism Is a Major Determinant of HIV-1 Reservoir Seeding in CD4 (+) T Cells and Offers an Opportunity to Tackle Infection. Cell Metab 29, 611-626 e615 (2019).
2. H. Pearson, Cancer patients opt for unapproved drug. Nature 446, 474-475 (2007).
3. S. Dhar, S. J. Lippard, Mitaplatin, a potent fusion of cisplatin and the orphan drug dichloroacetate. Proceedings of the National Academy of Sciences of the United States of America 106, 22199-22204 (2009).
4. S. Bonnet et al., A mitochondria-K+ channel axis is suppressed in cancer and its normalization promotes apoptosis and inhibits cancer growth. Cancer Cell 11, 37-51 (2007).
5. R. C. Sun et al., Reversal of the glycolytic phenotype by dichloroacetate inhibits metastatic breast cancer cell growth in vitro and in vivo. Breast Cancer Research and Treatment 120, 253-260 (2010).
6. M. Kato, J. Li, J. L. Chuang, D. T. Chuang, Distinct structural mechanisms for inhibition of pyruvate dehydrogenase kinase isoforms by AZD7545, dichloroacetate, and radicicol. Structure 15, 992-1004 (2007).
7. R. K. Pathak, S. Marrache, D. A. Harn, S. Dhar, Mito-DCA: A Mitochondria Targeted Molecular Scaffold for Efficacious Delivery of Metabolic Modulator Dichloroacetate. Acs Chem Biol 9, 1178-1187 (2014).
8. H. R. Christofk et al., The M2 splice isoform of pyruvate kinase is important for cancer metabolism and tumour growth. Nature 452, 230-U274 (2008).
9. L. H. Stockwin et al., Sodium dichloroacetate selectively targets cells with defects in the mitochondrial ETC. International Journal of Cancer 127, 2510-2519 (2010).
10. D. Kolb et al., Metabolic Modulation of the Tumor Microenvironment Leads to Multiple Checkpoint Inhibition and Immune Cell Infiltration. ACS Nano 14, 11055-11066 (2020).

1. A compound comprising a structure represented by Formula I, or a pharmaceutically acceptable salt thereof: 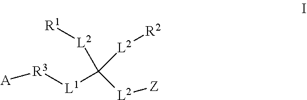
wherein
A comprises a mitochondrial targeting moiety;
Z comprises a lipid chain;
R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene, or —R^{a}X—, wherein R^{a} is a C₀-C₄ alkylene and X is a detectable-group.

2. The compound of claim 1, wherein the mitochondrial targeting moiety comprises a lipophilic delocalized cation.

3. The compound of claim 1, wherein the mitochondrial targeting moiety comprises an aryl phosphine or aryl phosphonium group (e.g., a monoaryl phosphonium, diaryl phosphonium, or triaryl phosphonium).

4. The compound of claim 1, wherein the mitochondrial targeting moiety comprises a phosphonium group (e.g., an aryl phosphonium group).

5. The compound of claim 1, wherein the mitochondrial targeting moiety comprises a substituted or unsubstituted triphenylphosphonium (TPP).

6. The compound of claim 1, wherein the compound comprises Formula II, or a pharmaceutically acceptable salt thereof: 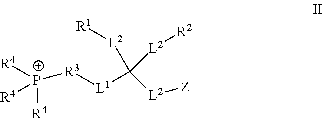
wherein
Z comprises a lipid chain;
R¹ and R² are independently hydrogen, a substituted or unsubstituted C₁-C₁₀ alkyl, a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
each R⁴ is independently a substituted or unsubstituted aryl or heteroaryl;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene, or —R^{a}X—, wherein R^{a} is a C₀-C₄ alkylene and X is a detectable-group.

7. The compound of claim 1, wherein the compound comprises Formula III, or a pharmaceutically acceptable salt thereof: 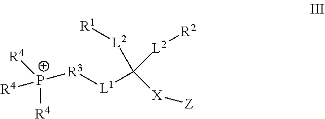
wherein
Z comprises a lipid chain;
R¹ and R² are independently hydrogen, a substituted or unsubstituted C₁-C₁₀ alkyl, or a glycolysis inhibitor, a glutamine oxidation inhibitor, or a fatty acid inhibitor, wherein at least one of R¹ or R² is a glycolysis inhibitor, a glutamine oxidation inhibitor, or a fatty acid inhibitor;
R³ is a substituted or unsubstituted C₁-C₁₀ alkylene;
each R⁴ is independently a substituted or unsubstituted aryl or heteroaryl;
L¹ is a substituted or unsubstituted alkylene, a carbonyl, an ester, an amide, a carbamate ester, an amine, an ether, a carbonate ester, a thioether, a thioester, or a urea;
each L² is independently a direct bond, a C₁-C₄ alkylene; and
X is a detectable-group.

8. The compound of claim 1, wherein at least one of R¹ or R² is lonidamine, dichloroacetate, alpha-tocopheryl succinate, methyl jasmonate, betulinic acid, and resveratrol, A-385358, ABT-263, ABT-737, AT-101, 2-amino-6-bromo-4-(1-cyano-2-ethoxy-2-oxoethyl)-4H-chromene-3-carboxylate (HA 14-1), LDH-A shRNA, orlistat, SB-204990, soraphen A, 4-(N-(s-glutathionylacetate)aminophenylarsenoxide (GSAO), clodronate, PK11 195, menadione, beta-lapachone, CD437, gamitrinibs, 8-(2-chloro-3,4,5-trimethoxybenzyl)-2-fluoro-9-(pent-4-nyl)-9H-purin-6-amine (PU24Fcl), (8-(6-bromobenzo[d][1,3,]dioxyl-5-ylthio)-9-(pent-4-nyl)-9H-purin-6-amine (PUH58), 8-(6-iodobenzo[d][1,3,]dioxyl-5-ylthio)-9-(3-isopropylamino)propyl-9H-purin-6-amine (PUH71), shepherdin, 2-methoxy estradiol, tetrathiomolybdate, buthionine sulphoximine, dimethylamino-parthenolide, parthenolide, imexons, magafodipir, menadione, motexafin gadolinium, PEITCs, elescomol (STA-4783), all trans-retinoic acid, 6-[3-(1-adamantyl)-4-hydroxyphenyl]-2-naphthalene carboxylic acid, E-3-(4′-hydroxy-3′-adamantylbiphenyl-4yl)acrylic acid, 3-bromopyruvate, butyric acid, 2-deoxyD-glucose, arsenite trioxide, or betulinic acid.

9. The compound of claim 1, wherein at least one of R¹ or R² is selected from the group consisting of:

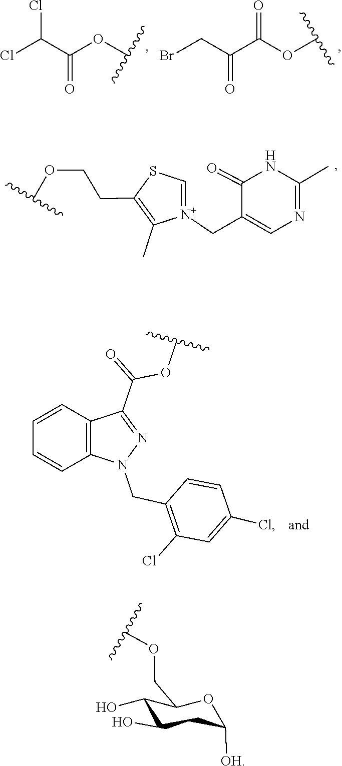

10. The compound of claim 1, wherein at least one of R¹ or R² is

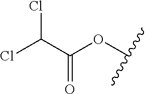 [SMILES: *OC(=O)C(Cl)Cl]

11. (canceled)

12. (canceled)

13. The compound of claim 1, wherein L¹ is an amide.

14. The compound of claim 1, wherein X is a silyl or a silyl ether.

15. The compound of claim 14, wherein the silyl ether is defined by the formula —O—Si R⁵R⁶—, wherein R⁵ and R⁶ are independently hydrogen or a C₁-C₈ alkyl.

16. The compound of claim 1, wherein the compound comprises Formula IV, or a pharmaceutically acceptable salt thereof: 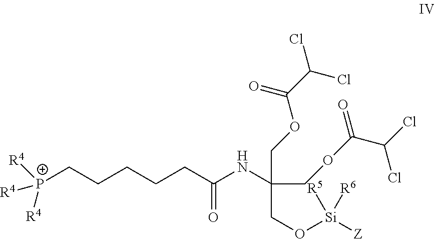
wherein
Z comprises a lipid chain;
each R⁴ is independently a substituted or unsubstituted aryl or heteroaryl; and
R⁵ and R⁶ are independently hydrogen or a C₁-C₈ alkyl.

17. (canceled)

18. The compound of claim 1, wherein Z is a substituted or unsubstituted C₅-C₂₅ alkyl.

19. The compound of claim 1, wherein Z is a C₁₀-C₂₅ linear alkyl.

20. The compound of claim 1, wherein the compound comprises: 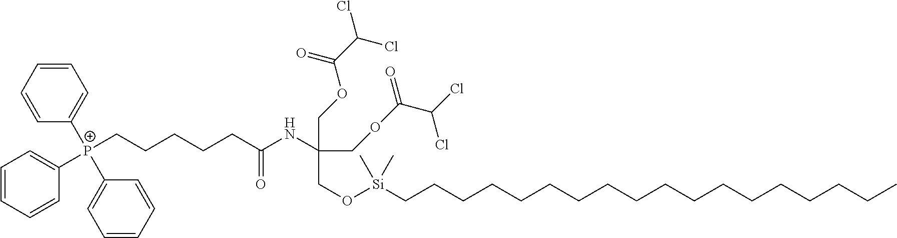 [SMILES: CCCCCCCCCCCCCCCCCC[Si](C)(C)OCC(COC(=O)C(Cl)Cl)(COC(=O)C(Cl)Cl)NC(=O)CCCCC[P+](c1ccccc1)(c1ccccc1)c1ccccc1]
or a pharmaceutically acceptable salt thereof.

21. A nanoparticle comprising:
the compound claim 1; and
a polyethylene glycol-lipid.

22. (canceled)

23. (canceled)

24. A pharmaceutical composition comprising a pharmaceutically acceptable carrier and an effective amount of the compound of claim 1.

25. (canceled)

26. (canceled)

27. A method of treating a disease or condition in a subject, the method comprising:
administering to the subject an effective amount of the compound of claim 1.

28-34. (canceled)
